(19) United States
(12) Patent Application Publication
(10) Pub. No.: US 2008/0057556 A1
(43) Pub. Date: Mar. 6, 2008

(54) Method of Increasing Amount of Insulin Produced and/or Secreted by Insulin-Producing Cell

(75) Inventors: Junji Miyakoshi, Aomori (JP); Tomonori Sakurai, Aomori (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: HIROSAKI UNIVERSITY, AOMORI (JP)

(21) Appl. No.: 11/570,835

(22) PCT Filed: Jun. 16, 2005
(86) PCT No.: PCT/JP05/11066

§ 371 Date: Dec. 18, 2006

(30) Foreign Application Priority Data
Jun. 18, 2004 (JP) 2004-180994
Mar. 1, 2005 (JP) 2005-056669

(51) Int. Cl. C12N 13/00

(52) U.S. Cl. 435/173.8

(57) ABSTRACT

[Problems] To provide a method of easily and efficiently increasing the amount of insulin produced and/or secreted at the cell level.

[Means for Solving Problems] The method is characterized in that an insulin-producing cell is exposed in vitro to an electromagnetic field with a frequency of 50 Hz±1 Hz or 60 Hz±1 Hz.

### TECHNICAL FIELD

The present invention relates to a method of increasing the amount of insulin produced and/or secreted by an insulin-producing cell.

### BACKGROUND ART

The phenomenon that an electromagnetic field with a frequency of 3 Hz to 300 Hz (an extremely low frequency alternating magnetic field: ELF-MF) has an influence on a living individual is well known. However, the whole picture of its action has not been fully understood yet, various studies have been made in order to elucidate it, and also an attempt has been made to apply the effect of the extremely low frequency alternating magnetic field on a living individual to the clinical medicine.

For example in Patent document 1, a method of controlling the blood glucose level in a patient with hyperglycemia by applying an electromagnetic wave with a resonant frequency of 15 Hz and an electromagnetic wave with a non-resonant frequency of 23 Hz to 28 Hz to the liver or the pancreas of the patient at a magnetic flux density of several militeslas has been proposed. Further, according to Non-patent document 1, in the case where an electromagnetic wave with a frequency of 10 Hz or 40 Hz is applied to a rat at a magnetic flux density of several militeslas, the serum insulin level is increased.

Patent document 1: U.S. Pat. No. 4,850,959

Non-patent document 1: Laitl-Koblerska et al., Influence of Alternating Extremely Low Frequency ELF Magnetic Field on Structure and Function of Pancreas in Rats, Bioelectromagnetics 23: 49-58 (2002)

### DISCLOSURE OF THE INVENTION

Problems that the Invention is to Solve

However, in either of the above-mentioned methods, the frequency to be employed is so special that an electromagnetic wave generator therefor is also special, therefore, they have a problem that it is difficult to simplify or downsize the device for practical application. Further, the phenomenon such as a decrease in the blood glucose level or an increase in the serum insulin level achieved by these methods was clearly the result of the effect of an electromagnetic wave on a living individual. However, when thinking of it at the cell level, as a matter of course, there is no clue how an electromagnetic wave acted on and caused such a phenomenon. Accordingly, these methods have a problem that they do not bring any useful information for applying it to the clinical medicine at the cell level.

Thus, an object of the present invention is to provide a method of easily and efficiently increasing the amount of insulin produced and/or secreted at the cell level.

Means for Solving the Problems

The present inventors made intensive studies in view of the above problems, and as a result, they found that when applying an electromagnetic wave with a commercial frequency of 50 Hz or 60 Hz among the extremely low frequencies to an insulin-producing cell in vitro, the amount of insulin produced and secreted by the insulin-producing cell is increased.

The present invention has been made based on the above-mentioned finding, and a method of increasing the amount of insulin produced and/or secreted by an insulin-producing cell of the present invention is characterized in that the insulin-producing cell is exposed in vitro to an electromagnetic field with a frequency of 50 Hz±1 Hz or 60 Hz±1 Hz as described in claim 1.

Further, the method described in claim 2 is characterized in that in the method described in claim 1, the insulin-producing cell during culture in a culture medium containing D-glucose is exposed to an electromagnetic field.

Further, the method described in claim 3 is characterized in that in the method described in claim 1, the insulin-producing cell during culture in a culture medium substantially free of D-glucose is exposed to an electromagnetic field and then cultured in a culture medium containing D-glucose.

Further, the method described in claim 4 is characterized in that in the method described in any one of claims 1 to 3, the magnetic flux density is set to a value from 0.5 mT to 50 mT.

Further, an insulin-producing cell of the present invention is characterized in that by exposing it in vitro to an electromagnetic field with a frequency of 50 Hz±1 Hz or 60 Hz±1 Hz, the amount of insulin produced and/or secreted is increased as described in claim 5.

### EFFECT OF THE INVENTION

According to the present invention, a method of easily and efficiently increasing the amount of insulin produced and/or secreted at the cell level is provided.

### BRIEF DESCRIPTION OF THE DRAWING

FIG. 1 is a schematic view of an extremely low frequency alternating magnetic field exposure unit that can be preferably used in the present invention.

### BEST MODE FOR CARRYING OUT THE INVENTION

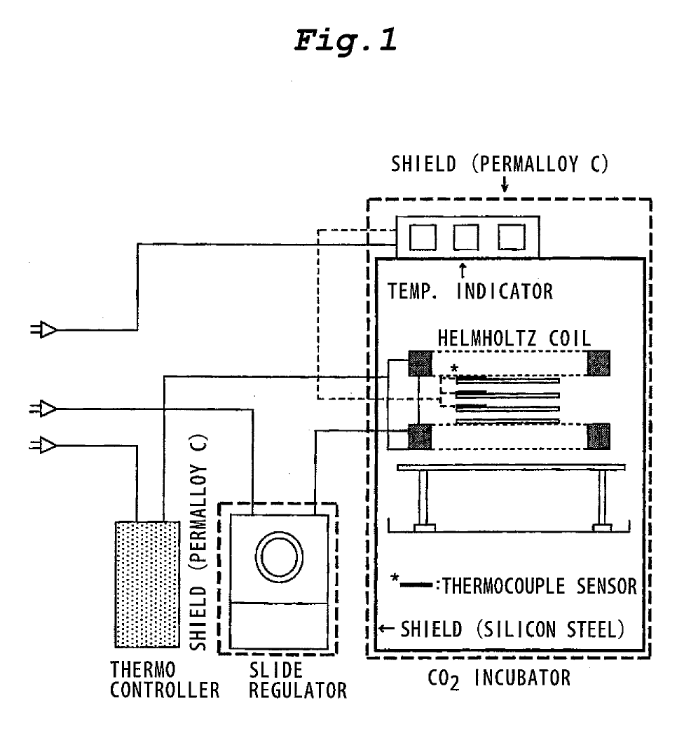

In the present invention, a method in which an insulin-producing cell is exposed in vitro to an electromagnetic field with a frequency of 50 Hz±1 Hz or 60 Hz±1 Hz is not particularly limited, and for example, it may be carried out by applying an electromagnetic wave with a frequency of 50 Hz or 60 Hz to an insulin-producing cell cultured in a culture dish. This procedure can be carried out by using, for example, an extremely low frequency alternating magnetic field exposure unit constructed in a CO₂ incubator described in Miyakoshi et al., J. Radiat. Res., 37: 185-191 (1996) (see FIG. 1)

In this case, the magnetic flux density is preferably set to a value from 0.5 mT to 50 mT, more preferably a value from 1 mT to 10 mT. When the magnetic flux density is less than 0.5 mT, there is a fear that a sufficient effect of the present invention may not be obtained, and when the magnetic flux density is more than 50 mT, there is a fear that the device may be complicated or the size of the device may increase, whereby its operation and maintenance may be difficult.

The application time of an electromagnetic wave to an insulin-producing cell is, for example, 10 minutes to 5 days. The application of an electromagnetic wave to an insulin-producing cell may be carried out continuously or intermittently.

In the method of increasing the amount of insulin produced and/or secreted by an insulin-producing cell of the present invention, as a preferred method, a method in which an insulin-producing cell during culture in a culture medium containing D-glucose (for example, a culture medium containing D-glucose at a concentration of from 0.1 g/L to 10 g/L, preferably from 0.5 g/L to 5 g/L) is exposed to an electromagnetic field and a method in which an insulin-producing cell during culture in a culture medium substantially free of D-glucose is exposed to an electromagnetic field and then cultured in a culture medium containing D-glucose (for example, a culture medium containing D-glucose at a concentration of from 0.1 g/L to 10 g/L, preferably from 0.5 g/L to 5 g/L) can be exemplified.

The insulin-producing cell to be applied in the present invention is not particularly limited as long as it is a cell having an ability to produce and/or secrete insulin, and as specific examples thereof, cells having an ability to produce and/or secrete insulin, which are differentiated from precursor cells, such as insulinoma cell lines, pancreatic islet cells, embryonic stem cells and somatic stem cells, transformed cells to which an ability to produce and/or secrete insulin is conferred by a cell engineering technique and the like can be exemplified. These cells may be any in terms of whether or not they are isolated and/or purified or how the purification degree is as long as they are outside a living organism by, for example, being extracted from a living individual using an appropriate method known per se. Further, insulin produced and/or secreted by the insulin-producing cell is not limited to a substance having a natural amino acid sequence, and may be a substance having an amino acid sequence with modification such as substitution, deletion or addition of an amino acid by a genetic engineering technique.

By using the insulin-producing cell in which the amount of insulin produced and/or secreted is increased according to the present invention as, for example, a cell for transplantation in pancreatic islet transplantation, which has been brought to attention as a new therapeutic method for type I diabetes recently, it is possible to try to reduce the number of cells required for the transplantation. Accordingly, the present invention contributes to the progress of the therapeutic method for type I diabetes by pancreatic islet transplantation. Further, the present invention can also be used as a method of improving the production efficiency when insulin is produced on a large scale as a drug product.

### EXAMPLES

Hereinafter, the present invention will be described in detail with reference to Examples, however, the invention should not be construed as being limited to the following description.

### Example 1

(Experimental Method)

A Syrian hamster-derived insulinoma cell line HIT-T15 was inoculated in a cell dish at 1.5×10⁵ cells/cm² and cultured in a RPMI1640 culture medium containing 10% fetal bovine serum and 1 g/L D-glucose. When the cells became 80% to 90% confluent by being cultured for 3 days, the culture medium was replaced with RPMI1640 culture media containing 10% fetal bovine serum and various concentrations of D-glucose and the cells were cultured while an electromagnetic wave with a frequency of 60 Hz is continuously applied at a magnetic flux density of 5 mT for 24 hours or 48 hours using an extremely low frequency alternating magnetic field exposure unit constructed in a CO₂ incubator shown in FIG. 1.

The amount of insulin contained in the culture medium after completion of the application was assayed by the ELISA method using an Insulin Assay kit manufactured by Shibayagi Co. Ltd., and the amount of insulin secreted by the cells was obtained. The amount of insulin secreted by the cells was standardized to the number of cells. The number of cells was counted by using a particle counter (manufactured by Beckman Coulter, Inc.).

Further, the cells were recovered from the culture media, and the recovered cells were treated with a solution whose composition was ethanol:12 normal hydrochloric acid:water=140:3:57 at −20° C. for 48 hours, whereby insulin in the cells was extracted. After the cells were pelleted down, the amount of insulin in the supernatant of the extract solution was assayed by the ELISA method using an Insulin Assay kit manufactured by Shibayagi Co. Ltd., and the amount of insulin in the cells was obtained. The amount of insulin in the cells was standardized to the total protein amount in the extract solution. The total protein amount in the extract solution was assayed with a Bio-Rad DC Protein Assay kit manufactured by Bio-Rad Laboratories, Inc., using a solution of bovine serum albumin as a standard substance.

(Experimental Result)

The amount of insulin secreted by the cells is shown in Table 1 and the amount of insulin in the cells is shown in Table 2. Further, in Table 1 and Table 2, the results of the cases where cells were cultured without exposure to the electromagnetic field are shown together (pseudo-exposure culture group). As is obvious from Table 1 and Table 2, it was found that, according to this method, the amount of insulin produced and/or secreted by the insulin-producing cell could be increased.

TABLE 1

| D-glucose | | Amount of Insulin secreted by cell | |
|---|---|---|---|
| concentration in | | Electromagnetic field | Pseudo-exposure |
| culture | Culture | exposure culture group | culture group |
| medium (g/L) | time (H) | (μU/10⁶ cells) | (μU/10⁶ cells) |
| 0 | 48 | 2200 | 1600 |
| 1.0 | 24 | 12800 | 8200 |
| | 48 | 13000 | 9600 |
| 2.0 | 24 | 12600 | 8000 |
| | 48 | 16800 | 14300 |
| 4.5 | 24 | 17600 | 13500 |

TABLE 2

| D-glucose | | Amount of Insulin in cell | |
|---|---|---|---|
| concentration in | | Electromagnetic field | Pseudo-exposure |
| culture | Culture | exposure culture group | culture group |
| medium (g/L) | time (H) | (μU/mg protein) | (μU/mg protein) |
| 1.0 | 24 | 5600 | 4800 |
| 2.0 | 24 | 7000 | 6600 |
| | 48 | 8300 | 6800 |
| 4.5 | 24 | 8300 | 7400 |

### Example 2

It was confirmed that also in the case where an electromagnetic wave with a frequency of 50 Hz is applied to an insulin-producing cell, the same effect as in Example 1 could be obtained.

### Example 3

(Experimental Method)

A Syrian hamster-derived insulinoma cell line HIT-T15 was inoculated in a cell dish at 1.5×10⁵ cells/cm² and cultured in a RPMI1640 culture medium containing 10% fetal bovine serum and 1 g/L D-glucose. When the cells became 80% to 90% confluent by being cultured for 3 days, the culture medium was replaced with a RPMI1640 culture medium containing 10% fetal bovine serum and free of D-glucose and the cells were cultured while an electromagnetic wave with a frequency of 60 Hz is continuously applied at a magnetic flux density of 5 mT for 30 minutes or 2 hours using an extremely low frequency alternating magnetic field exposure unit constructed in a CO₂ incubator shown in FIG. 1.

Immediately after or 2 hours after completion of the application, the culture medium was replaced with a RPMI1640 culture medium containing 10% fetal bovine serum and 3 g/L D-glucose, and the cells were cultured for 2 hours in a standard CO₂ incubator, whereby glucose stimulation was carried out. The amount of insulin contained in the culture medium after completion of the culture was assayed by the ELISA method using an Insulin Assay kit manufactured by Shibayagi Co. Ltd., and the amount of insulin secreted by the cells was obtained. The amount of insulin secreted by the cells was standardized to the number of cells. The number of cells was counted by using a particle counter (manufactured by Beckman Coulter, Inc.).

(Experimental Result)

The amount of insulin secreted by the cells is shown in Table 3. Further, in Table 3, the results of the cases where cells were cultured without exposure to the electromagnetic field and then the glucose stimulation was carried out are shown together (pseudo-exposure culture group). As is obvious from Table 3, it was found that, according to this method, the amount of insulin produced and/or secreted by the insulin-producing cell could be increased.

TABLE 3

| | | | Amount of Insulin secreted by cell | |
|---|---|---|---|---|
| Electromagnetic | | Time until culture medium | Electromagnetic field | Pseudo-exposure |
| field exposure | | was replaced after | exposure culture group | culture group |
| time | | completion of application | (μU/10⁶ cells) | (μU/10⁶ cells) |
| 2 | hours | Immediately | 14.5 | 11.8 |
| 2 | hours | 2 hours | 22.4 | 14.6 |
| 30 | minutes | Immediately | 29.4 | 19.1 |

### Example 4

It was confirmed that also in the case where an electromagnetic wave with a frequency of 50 Hz is applied to an insulin-producing cell, the same effect as in Example 3 could be obtained.

### INDUSTRIAL APPLICABILITY

The present invention has industrial applicability in the point that it can provide a method of easily and efficiently increasing the amount of insulin produced and/or secreted at the cell level.

1. A method of increasing the amount of insulin produced and/or secreted by an insulin-producing cell, characterized in that the insulin-producing cell is exposed in vitro to an electromagnetic field with a frequency of 50 Hz±1 Hz or 60 Hz±1 Hz.

2. The method according to claim 1, characterized in that the insulin-producing cell during culture in a culture medium containing D-glucose is exposed to an electromagnetic field.

3. The method according to claim 1, characterized in that the insulin-producing cell during culture in a culture medium substantially free of D-glucose is exposed to an electromagnetic field and then cultured in a culture medium containing D-glucose.

4. The method according to any one of claims 1 to 3, characterized in that the magnetic flux density is set to a value from 0.5 mT to 50 mT.

5. An insulin-producing cell, characterized in that by exposing it in vitro to an electromagnetic field with a frequency of 50 Hz±1 Hz or 60 Hz±1 Hz, the amount of insulin produced and/or secreted is increased.
